(19) United States
(12) Patent Application Publication
(10) Pub. No.: US 2004/0073296 A1
(43) Pub. Date: Apr. 15, 2004

(54) Inhibition of restenosis using a DNA-coated stent

(76) Inventors: Stephen E. Epstein, Rockville, MD (US); Shmuel Fuchs, Rockille, MD (US)

Correspondence Address: HELLER EHRMAN WHITE & MCAULIFFE LLP, 1666 K STREET,NW, SUITE 300, WASHINGTON, DC, 20006, US

(21) Appl. No.: 10/457,019

(22) Filed: Jun. 9, 2003

Related U.S. Application Data
(63) Continuation of application No. PCT/US01/45755, filed on Dec. 7, 2001.
(60) Provisional application No. 60/251,579, filed on Dec. 7, 2000.

(51) Int. Cl. A61F 2/06

(52) U.S. Cl. 623/1.46

(57) ABSTRACT

Restenosis of arteries after angioplasty is inhibited by implanting in the treated artery a stent incorporating genes that encode gene products having anti-restenotic activity. The genes may be incorporated into a coating on the stent structure or in cells that are affixed to the stent. The genes or cells containing them may be adhered to the struts of the stent or incorporated in a collagen matrix that forms a coating covering the struts and interstices of the stent

### RELATIONSHIP TO OTHER APPLICATIONS

This application claims the benefit of the priority of copending U.S. Provisional Patent Application No. 60/251,579, filed Dec. 8, 2000.

### BACKGROUND OF THE INVENTION

1. Field of the Invention

This invention relates to preventing restenosis of arteries after angioplasty and more particularly to use of a stent platform to deliver gene products through DNA or transfected cells that have been incorporated into a coating applied to the stent, the gene products of which will prevent such restenosis

2. Brief Description of the Prior Art

Coronary angioplasty has become an important method of treating narrowed (stenotic) arteries supplying the heart or the legs. Although the initial success rate of coronary angioplasty for opening obstructed coronary arteries exceeds 95%, restenosis occurs at the site of angioplasty in 25-50% of patients within six months, regardless of the type of angioplasty procedure used. Although the use of stents has appreciably reduced the rate of restenosis, even with this treatment strategy restenosis occurs in 5 to 20% of patients. Importantly, when restenosis occurs within a stent, the chance that restenosis will recur is very high. Thus, the problem of restenosis is still formidable, despite recent advances in reducing its incidence.

Two primary mechanisms appear to be involved in the development of restenosis. First, recoil of the vessel wall (negative remodeling) leads to gradual narrowing of the vessel lumen. Second, an exaggerated healing response of medial and/or adventitial smooth muscle cells (SMCs) to vascular injury, which involves the excessive proliferation of SMCs and the migration of SMCs to the subintima, where they continue to proliferate and begin to secrete extracellular matrix. These processes involving SMCs cause the neointimal mass to expand and gradually encroach upon the coronary lumen. Ultimately the expanding lesion narrows the vessel, increases resistance to blood flow, and causes ischemic symptoms. In the absence of stenting, both remodeling and an expanding neointima contribute to restenosis. When stents are deployed negative vascular remodeling is prevented and restenosis occurs only as a result of the expanding neointimal mass. Given these pathophysiologic mechanisms, the problem of controlling restenosis occurring with stent deployment becomes largely the problem of controlling the development of the neointimal mass.

Many attempts have been made to prevent the development of restenosis. Although many have been reported to be successful in inhibiting neointima development in various experimental models, almost invariably their translation to clinical interventions has been without success. These strategies have included the oral administration of drugs, their systemic administration, and their local delivery.

Local Delivery:

Therapeutic strategies began to focus on local delivery, as it became apparent that high concentrations of active agent were needed at the target site. It would be very unlikely that such high concentrations could be achieved by any other approach than local delivery. Unfortunately, despite years of development and testing, the consensus is that catheter delivery systems are too inefficient to provide a high probability of success. Only one percent or less of the delivered product appears to persist for any period of time in the vessel wall.

Coated Stents to Deliver Proteins or Small Molecules:

The concept that drugs could be incorporated into the coating of stents has become popularized, with mixed results. Most studies have shown no effect. However, preliminary encouraging results using stents having a coating impregnated with either Taxol or its derivatives, or Rapamycin, have been reported at several international meetings. Although this strategy may ultimately prove to be successful with specific drugs, one of the possible problems is that proteins and small molecules have short therapeutic half-lives. They may undergo degradation such that proper concentrations at the target area will not be achieved, or not be achieved for a long enough time to attain anti-restenosis activity. This situation makes stent release kinetics critical, because it is solely the ability of the stent coating to release proper concentrations of active agent over a period of at least several weeks that will determine the success of the intervention. This raises an important practical problem inherent with most current coatings, i.e., existing polymers must be tailored to each protein or small molecule that is being tested for anti-restenosic activity, thereby making it extremely difficult and labor-intensive to design appropriate coatings for each different candidate drug.

Another problem with existing coating polymers is that they may degrade any DNA (genes) incorporated into them. An additional problem is that if the coating is to contain transfected cells expressing anti-restenosis gene products, existing polymers may be toxic to such cells.

Finally, the metallic surface of stents occupies only about 15-20 percent of the total area subsumed by the stent. The rest of the area consists of open space. Most coatings are applied to the metal struts of the stent, leaving the interstices free of coating. This poses what could be a formidable problem; it means that 80-85% of the vessel wall to which the stent is apposed will not directly contact the therapeutic agent, or the cell expressing a-potentially therapeutic gene product.

Thus, a number of problems can be foreseen in such attempts to deliver drugs and the like by means of coated stents and a number of problems can be foreseen in attempts to deliver DNA or cell-based delivery of agents using existing stent coatings.

It is often necessary to design or formulate a polymer specifically for each individual therapeutic drug in order to achieve optimal release kinetics.

It is necessary to design or formulate each polymer to avoid degradation or interaction with the drug or protein incorporated therein

Existing stent coatings may degrade any incorporated DNA.

Existing stent coatings may be toxic to incorporated transfected cells.

The area of stent coating/vessel wall contact is limited to 15-20% of the area subsumed by the stent.

A number of studies have been published relating to stent-based anti-restenosis therapy employing various drugs in stent coatings to achieve anti-restenosis effects. Certain publications, discussed below, have dealt with delivery of genes in association with an intravascular stent.

Feldman, M. D. et al., “Stent-based Gene Therapy”, J. Long Term Eff. Med. Implants 2000, 10(1-2):47-68, report the use of a stent having microneedles. They evaluated a gene-stent delivery mechanism based on microporous metal microneedles developed with nanotechnology in an attempt to overcome some of these problems. These authors evaluated transfection of genes by microfabricated technology in smooth muscle cells in culture. They demonstrated that microneedles can deliver gene therapy to smooth muscle cells in culture and can produce controlled penetration of the IEL and intima. They concluded that taller microneedles need to be developed to reach the media in diseased human arteries and that this technology has the potential to be incorporated in a stent to deliver gene therapy in atherosclerotic plaque. Thus, the concept of Feldman et al was to develop a stent with very small needles (“microneedles”) to inject genes directly into cells of the vessel wall.

Van Belle, E., et al. “Passivation of metallic stents after arterial gene transfer of phVEGF165 inhibits thrombus formation and intimal thickening”, J. Am. Coll. Cardiol. 1997, May:29(6):1371-1379, investigated whether direct gene transfer of an endothelial cell mitogen could passivate metallic stents by accelerating endothelialization of the prosthesis. Naked plasmid DNA encoding vascular endothelial growth factor (VEGF) was delivered locally using a hydrogel-coated balloon angioplasty catheter to 16 rabbit iliac arteries in which metallic stents had been placed at the site of balloon injury. Thus, the concept of Van Belle et al. was to separately place plasmid DNA into the wall of a vessel, using a special cathether, and then to deploy a stent. The stent was not used to deliver anti-restenosis agents.

Dichek, D. A., et al., “Seeding of intravascular stents with genetically engineered endothelial cells”, Circulation 1989, November 80(5):1347-1353, seeded stents with genetically engineered endothelial cells in vitro. The endothelial cells were seeded onto stainless steel stents and grown until the stents were covered. Their intention was to provide a solution to the recognized problems of local thrombosis and restenosis due to intimal proliferation. In this study, only the struts of the stent were covered with cells, and no coating was provided to facilitate adherence of the cells to the stent.

Accordingly, a need has continued to exist for improved methods of preventing restenosis of arteries after angioplasty and for improved stents to assist in accomplishing this goal.

### SUMMARY OF THE INVENTION

An advance in the treatment of restenosis after angioplasty has been achieved by the invention wherein a stent is implanted in the treated artery incorporating genes that encode gene products with anti-restenotic activity. The genes may be incorporated into a coating on the stent structure or in cells that are affixed to the stent.

Accordingly, it is an object of the invention to provide a method for preventing or alleviating restenosis of an artery after angioplasty.

A further object is to provide a stent for implantation into an artery after angioplasty that is coated with at least one gene coding for an anti-restenotic factor.

A further object is to provide a stent coated with cells containing genes producing anti-restenotic gene products. Further objects will be apparent from the description of the invention which follows.

### BRIEF DESCRIPTION OF THE DRAWINGS

FIG. 1A illustrates an uncoated or bare stent of the type implanted in an artery after angioplasty to inhibit restenosis.

FIG. 1B is a schematic illustration of the stent of FIG. 1A coated with DNA.

FIG. 1C is a schematic illustration of an enlarged portion of the coated stent of FIG. 1B. The base of the figure is a cross-section through the stent. The irregular lattice-work of hoop-like structures represents the polymer of the stent coating, which has plasmid DNA incorporated into it (small dots).

FIG. 1D is a schematic illustration of the stent of FIG. 1A coated with DNA suspended in a collagen gel, which is held in place by the lattice-work of polymer hoops.

FIG. 1E is a schematic cross-section of a portion of the stent of FIG. 1D and adjacent artery wall showing the DNA suspended in a layer of collagen gel, which is held in place by the lattice-work of polymer hoops

FIG. 2A illustrates an uncoated or bare stent of the type implanted in an artery after angioplasty to inhibit restenosis.

FIG. 2B is a schematic illustration of the stent of FIG. 2A having transformed endothelial cells implanted on the surface of its struts. The cells are incorporated into the irregular lattice-work of hoop-like structures depicted in FIG. 1C, which represents the polymer of the stent coating.

FIG. 2C is a schematic illustration of an enlarged view of a cross-section of a portion of the stent of FIG. 2A having a layer of collagen gel containing implanted transformed endothelial cells, which are held in place by the lattice-work of polymer hoops.

### DETAILED DESCRIPTION OF THE INVENTION AND PREFERRED EMBODIMENTS

According to the invention a stent for implantation into an artery after angioplasty is coated with genes that code for products that inhibit restenosis of the treated artery or with transformed cells containing such genes.

A number of therapeutic strategies may be used for supplying the arterial wall with anti-restenosis factors coded by the genes.

In a first embodiment or strategy of the invention, plasmid DNA or viral vector is incorporated into a stent coating, which comprises a substance that adheres to the stent and incorporates the DNA or viral vector, or transformed cells, without damaging them. Thereby the coating facilitates DNA delivery to, and transfection of, cells within the injured vessel wall, or cells that are migrating from the media and/or adventitia to form the neointima. The genes within the stent coating will encode gene products with anti-restenosis activities. The coating can be formed from any material that can cover the surface of the stent and that has the above characteristics. One such candidate coating has been created by the Photolink® process of the SurModics company (Eden Prairie, Minn.).

Within the first embodiment or strategy of the invention, two alternatives may be used:

1. DNA is incorporated in the stent coating, covering stent struts but not intervening spaces.

2. The stent coating will act as a support scaffolding for the binding of collagen to the stent. The collagen will providea matrix for the DNA that will allow complete coverage of the vessel wall. An example of such a collagen matrix (but not limited to this particular one) is the collagen matrix manufactured by Selective Genetics. The collagen matrix will facilitate two important features of the invention.

a. It will provide efficient contact between DNA and all of that part of the vessel in which the stent is deployed so that a greater percentage of the cells within the vessel wall will be in tight apposition to the DNA, thereby enhancing DNA incorporation and expression of the therapeutic transgene.

b. It will provide a “DNA/collagen barrier” to cells migrating from the media or adventitia on their way to form the expanding neointima. These cells, as they pass through the DNA/collagen barrier will transiently reside in a perfect anatomic milieu for efficient transfection (or infection).

The intimate contact between the DNA (whether plasmid DNA or viral vector containing the therapeutic transgene) within the stent coating and vessel wall cells leads to efficient incorporation of the DNA into these cells. The subsequent expression of transgene product will exert the desired therapeutic effects on these cells, such as (but not limited to) inhibition of smooth muscle cell (SMC) proliferation or migration, induction of SMC apoptosis, or inhibition of the inflammatory response to vessel injury.

Those skilled in the art will recognize that, although collagen is currently a preferred matrix for suspending the DNA or vectors, other polymeric matrices capable of suspending the DNA or viral vectors and of filling the interstices between the struts of the stent can be used, provided that they exhibit the necessary compatibility with the DNA or viral vector and permit release of the active agents to the adjacent artery wall or to cells migrating through the matrix. The properties of many such natural or synthetic polymeric matrices are well known or can be determined without undue experimentation to determine their suitability for use in the stent of this invention.

The use of a stent coated with a DNA capable of transfecting cells so they produce anti-restenotic factors by introduction of one or more genes coding for such products provides one solution to the problem of the short half-lives of the anti-restenotic agents introduced as proteins. Once a cell is transfected with a gene encoding a gene product with anti-restenosis activities, it will express that protein for extended periods of time. The target cell to be transfected could be the smooth muscle cells present in the vessel media and or adventitia, i.e., the cell destined to migrate to the neointima and be the dominant cell contributing to the expanding neointima. Alternatively, autologous cells could be transfected ex vivo, and incorporated into the coating of a stent. Such a cell would then express and secrete its anti-restenosis transgene product over several weeks, exerting inhibitory effects on those cells of the vessel wall involved in the restenosis process. Thus, the invention eliminates the problem presented by the short half-lives of therapeutic proteins. The transfected cells will continually express their transgenes for as long as the transfected DNA remains functionally intact within the transfected cell, usually longer than 2-3 weeks. Endothelial cells themselves could express multiple products that exert anti-restenotic activities.

According to the invention it would not be necessary to redesign a carrier polymer for each protein or small molecule used for anti-restenotic therapy in order to provide for optimal release kinetics, because the transformed cells will continue to produce and release the therapeutic materials for an extended period of time. The use of DNA or transduced cells as part of the delivery system also permits the administration of more than one treatment agent, because multiple different DNAs or transduced cells, each causing the expression of a different transgene, can be incorporated into a single stent delivery platform. Because of the complexity of the release kinetics of stent coatings, it is difficult to incorporate different proteins or small molecules into the coating of a stent.

Another advantage of the invention is that the composition of the coating material can be tailored to preserve and support the DNA or cells to be incorporated into the coating. The material should, of course, not degrade the incorporated DNA. However, the design and formulation of the coating material is nevertheless simplified because it does not have to accommodate a wide variety of proteins and/or small molecules.

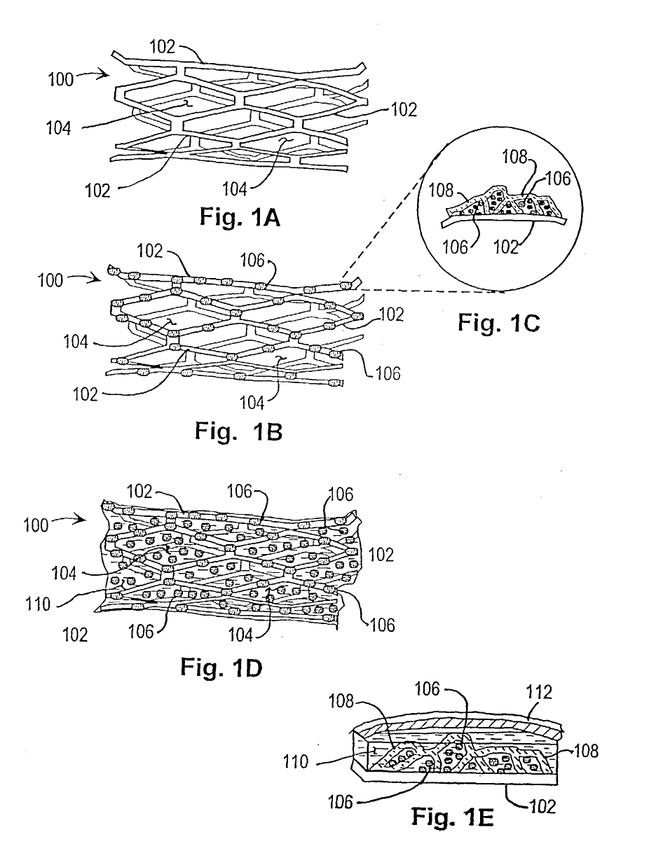

The stent used in the first embodiment of the invention is illustrated in FIGS. 1A-1E of the drawings. These figures illustrate a stent coated with DNA by incorporating plasmid DNA or a viral vector into a coating material that adheres to the stent (with or without a collagen gel) and into which DNA (as plasmid or viral vector) can be incorporated. The coated stent facilitates DNA delivery to, and transfection of, cells within the injured vessel wall, or cells that are migrating from the media and/or adventitia to form the neointima. The genes within the stent coating will be selected or created to encode gene products with anti-restentosis activities.

FIG. 1A illustrates the bare stent 100 without coating and without DNA or viral vectors. The stent comprises struts 102 having interstices or openings 104 between them.

FIG. 1B illustrates the stent 100 with a coating that has plasmid DNA or viral vectors 106 incorporated into it. The coating and its contained genes cover the metal struts 102 but not the intervening spaces 104 FIG. 1C is a greatly enlarged view of a cross-section of a portion of the stent 100 of FIG. 1B, as indicated by the guidelines, showing the coated struts 102 with associated DNA 106. The lower portion of the figure shows a cross-section of a strut 102 of the stent 100. The irregular lattice work of hooplike structures 108 represents the polymer of the stent coating, which has plasmid DNA 106 (small dots) incorporated therein.

FIG. 1D illustrates the stent 100 of FIG. 1A provided with a coating of collagen 110 containing plasmid DNA or viral vectors 106. The stent 100, with its lattice-work of polymer hoops 108, serves as a scaffold for supporting the collagen gel 110 that has plasmid DNA or viral vectors 106 incorporated into it. The coating of the collagen gel 108 with contained genes 106 supported by the stent 100 covers not only the metal struts 102 (which cover only 15-20% of the arterial wall over which the stent extends), but also the intervening spaces 104, providing total coverage of the arterial wall.

FIG. 1E is a greatly enlarged cross-sectional side view of the stent 100 shown in FIG. 1D. It can be seen that the stent 100 incorporating a collagen gel layer 110 provides a “DNA/collagen barrier” to cells migrating from the media or adventitia of the arterial wall 112 on their way to form the expanding neointima. These cells, as they pass through the DNA/collagen barrier 110, will transiently reside in a perfect anatomic milieu for efficient DNA transduction. The collagen gel 110 is held in place by the lattice-work of polymer hoops 108.

In a second embodiment or therapeutic strategy of the invention, progenitor endothelial cells transduced with therapeutic transgenes are incorporated into a stent coating. The coating comprises a substance that adheres to the stent and incorporates the cells without damaging them. The implanted endothelial cells will have been transfected (or infected) ex vivo, with vectors containing transgenes encoding gene products with anti-restenosis activities. This anatomic platform facilitates exposure of cells within the injured vessel wall (or cells that are migrating from the media and/or adventitia to form the neointima) to the therapeutic gene product expressed by the endothelial cells.

As with the first invention strategy or embodiment, this variant of the invention can employ any coating that can be attached to a stent and that has the above characteristics. One such candidate coating has been created by the Photolink® process of the SurModics Company (Eden Prairie, Minn.).

The therapeutic concept on which this variant of the invention is based is as follows. The transfected progenitor endothelial cells will express and secrete their therapeutic transgene product for a prolonged time (at least 2-3 weeks). Moreover, it will be secreted directly into the apposed vessel wall, resulting in high local concentrations of transgene product that will stand an excellent chance of exerting the desired therapeutic effects on these cells, such as (but not limited to) inhibition of smooth muscle cell (SMC) proliferation or migration, induction of SMC apoptosis, or inhibition of the inflammatory response to vessel injury.

Within the second embodiment or strategy of the invention two alternatives may be used:

1. Progenitor endothelial cells may be put into the stent coating itself, which will cover the metal struts but not the intervening spaces.

2. The stent coating will act as a support scaffolding for binding the collagen to the stent. The collagen will provide a matrix for the cells that will allow complete coverage of the vessel wall. This will facilitate two important features of the invention.

a. It will provide efficient contact between progenitor endothelial cells and vessel wall cells so that a greater percentage of the cells within the vessel wall will be exposed to high concentrations of the therapeutic gene product.

b. It will provide an “endothelial cell/collagen barrier” to vessel wall cells that are migrating from the media or adventitia on their way to form the expanding neointima. These cells, as they pass through the endothelial cell/collagen barrier, will transiently reside in a perfect anatomic milieu for exposure to high concentrations of the therapeutic gene product.

As discussed above, other natural or synthetic polymers having appropriate properties can be used in place of the collagen.

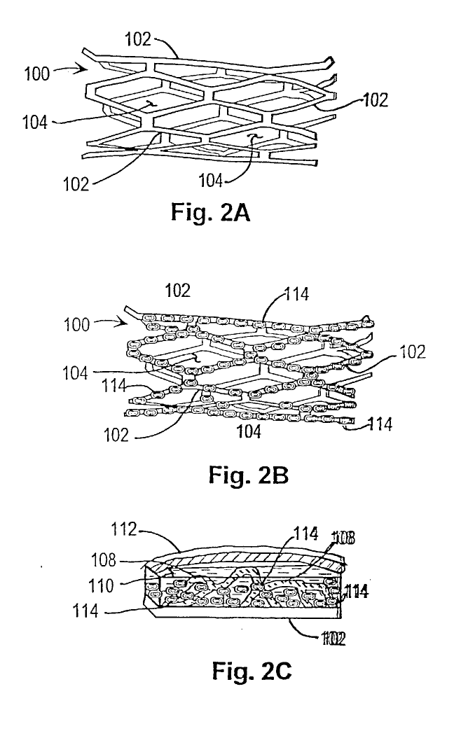

The stent used in the second embodiment of the invention is illustrated in FIGS. 2A-2C of the drawings. These figures illustrate coating a stent with cells by incorporating them into a stent coating, which comprises a substance that adheres to the stent (with or without a collagen gel) and into which cells can be incorporated. As in the first embodiment of the invention, the coated stem of the second embodiment facilitates DNA delivery to, and transfection of, cells within the injured vessel wall, or cells that are migrating from the media and/or adventitia to form the neointima. The genes within the cells incorporated into the stent coating will be selected or created to encode gene products with anti-restentosis activities.

FIG. 2A illustrates the bare stent 100, having struts 104 and openings or interstices 104, without coating and without affixed cells.

FIG. 2B illustrates the stent 100 with a coating that has cells 114 incorporated into it. The cells 114 have been transduced with genes encoding proteins with therapeutic anti-restenosis activities. The coating and its contained cells 114 cover the metal struts 104 of the stent 100 but not the intervening spaces 104. The cells are incorporated into an irregular lattice-work of hoop-like structures similar to those depicted in FIG. 1C as polymer loops 108, which represent the the polymer of the stent coating. The cells can also be incorporated into a stent having a layer of collagen gel 110 analogous to that illustrated for the first embodiment of the invention in FIG. 1D.

FIG. 2C illustrates a greatly enlarged side view cross-section of such a stent 100 having a collagen gel coating layer 110 wherein the stent 100, with its coating of a lattice-work of polymer hoops 108, serves as a scaffold for supporting the collagen gel layer 110. The collagen gel layer 110 incorporates transduced endothelial cells 114.

The coating and the collagen gel it supports contain cells that cover not only the metal struts 102 (which cover only 1520% of the arterial wall over which the stent extends), but also the intervening spaces 104, providing total coverage of the arterial wall 112. Consequently, the collagen gel coating 110 of the stent 100 provides an “endothelial cell/collagen barrier” to cells migrating from the media or adventitia of the arterial wall 112 on their way to form the expanding neointima. The arterial wall cells, as they pass through the endothelial cell/collagen barrier, will transiently reside in a perfect anatomic milieu for efficient exposure to anti-restenosis agents expressed by the transduced cells.

In view of the above disclosure it will be understood that the invention involves systems to deliver to cells of an injured vessel wall genes and/or autologous transfected endothelial cells to deliver gene products to the injured vessel wall. This delivery of genes and/or gene products is accomplished by implanting into an artery treated by angioplasty a stent having a coating, with or without a collagen matrix, containing the genes or transfected endothelial cells. The embodiments of the delivery system of the invention using a collagen matrix, will have the added advantage of providing a DNA/collagen barrier, or endothelial cell/collagen barrier, that will both retard migration of cells to the developing neointima and, more importantly, will provide an extremely efficient means of exposing the migrating cells to the therapeutic genes or gene products. The strategy of using DNA or transduced cells as part of the delivery system will give added versatility to the method and apparatus of the invention, as it will allow for multiple sets of DNA or cells, each expressing a different transgene, to be incorporated into the stent delivery platform. Because of the complexity of the release kinetics of stent coatings, it is difficult if not impossible to incorporate different proteins or small molecules into the coating of a stent.

Accordingly, the invention provides the benefits of substantially reducing the incidence of restenosis with minimal incidence of untoward complications, a result that has been achieved to only a limited extent (or, as with radiation therapy, carrying unknown future risk) with other anti-restenosis strategies.

Methods and Materials Used in Practicing the Invention:

A. Therapeutic Agents.

The therapeutic agents used in this invention can be any gene encoding a protein that has been demonstrated to have, or is suspected of having, anti-restenosis effects. Examples include, but are not limited to, endostatin and angiostatin. Other examples include, but are not limited to, genes that encode a product that inhibits the effects of known or as yet unknown agents that facilitate restenosis, by either binding to the agent and preventing its activity, by binding to its receptor, or by inhibiting any aspect of the signaling cascade initiated by the binding of the agent to its receptor. Examples of targets for anti-restenosis strategies would include, but not be limited to VEGF, its receptors, and its signaling cascade; and bFGF, its receptors, and its signaling cascade.

B. Obtaining Autologous Progenitor Endothelial Cells.

Progenitor Endothelial Cells: There are at least two potential sources for the progenitor endothelial cells that will be incorporated into the stents, i.e., the circulating blood and the bone marrow.

Peripheral blood mononuclear cells: The most common method of obtaining endothelial progenitor cells is to isolate them from among peripheral blood mononuclear cells (PBMCs). PBMCs are isolated from clotted blood by density gradient centrifugation with Histopaque-1077 (Sigma). Cells are plated on coated culture dishes (Sigma) and maintained in medium designed for optimal growth of endothelial cells. After culturing for several days, nonadherent cells are removed by washing with PBS, new media is applied, and the cells are maintained in culture for 7-10 days.

Bone marrow: An alternate method for isolating progenitor endothelial cells is to culture them from autologous bone marrow. With this approach bone marrow is aspirated from the patient who is to receive stent implantation using standard clinical techniques. Bone marrow (BM) cells are harvested under sterile conditions in preservative free heparin (20 units/mil BM cells) and filtered sequentially using 300% and 212μ stainless steel mesh filters. BM cells are then isolated by Ficoll Hypaque gradient centrifugation and cultured in long-term culture medium (LTCM) (Stem Cell Tech, Vancouver, British Columbia, Canada) at 33° C. with 5% CO₂, in a T-75 culture flask. It will be understood by those skilled in the art that, although the specification discloses two specific methods to isolate progenitor endothelial cells, i.e., from PBMCs and from autologous bone marrow, the invention does not exclude the use of any alternative method that may be found useful to provide cells useful in the practice of the invention.

To assure that the cultured cells are progenitor endothelial cells, at least two assays are performed on an aliquot of the cells.

Staining of progenitor endothelial cells: Fluorescent detection of progenitor endothelial cells will be performed by using direct fluorescent staining to detect dual binding of FITC-labeled Ulex europaeus agglutinin (UEA-I) (Sigma) and 1,1′-dioctadecyl-3,3,3′,3′-tetramethylindocarbocyanine (DiI)-labeled acetylated low density lipoprotein (acLDL; Biomedical Technologies, Stoughton, Mass.). Attached PBMCs after 7-10 days in culture are incubated with acLDL at 37° C. and then fixed with 1% paraformaldehyde for 10 min. After washes, the cells will be exposed to UEA-1 (10 μg/ml) for 1 hour. Cells identified as having double-positive fluorescence will be classified as differentiating progenitor endothelial cells.

Fluorescence-Activated Cell Sorting Fluorescence-activated cell sorting (FACS) detection of progenitor endothelial cells is performed on cells detached with trypsin and/or PBS with 1 mM EDTA. Cells (2×10⁵) are incubated for 30 min at 4° C. with the monoclonal antibodies targeted to epitopes specific for endothelial cells, such as the KDR receptor. After incubation, the cells will be fixed in 1% paraformaldehyde and quantitative FACS performed.

Those skilled in the art will recognize that, although the specification discloses progenitor endothelial cells by way of a particular and exemplary embodiment, the invention does not exclude the use of any alternative cell type that can provide the benefit of inhibiting restenosis. Alternative cell types may be discovered, and may even be found to be superior to progenitor endothelial cells for use in the context of this invention. Such superiority could be manifested in several, non-exclusive, ways. Such cells might be easier to obtain, e.g., non-immunogenic non-autologous cells, or cells derived from the patient's skin, etc Such cells might be easier to incorporate into the stent coating, might have characteristics that permits greater ease of transfection, and/or might exhibit greater efficiency of gene expression. All such alternative cell types are to be considered as included within the invention.

From the above disclosure it can be seen that the invention has a number of advantages over the currently used techniques for inhibiting restenosis.

The invention eliminates the critical nature of redesigning a polymer for each protein or small molecule so that optimal release kinetics are achieved. The strategy of using DNA or transduced cells as part of the delivery system will allow for multiple sets of DNA or cells, each expressing a different transgene, to be incorporated into the stent delivery platform. Because of the complexity of the release kinetics of stent coatings, it is difficult if not impossible to incorporate different proteins or small molecules into the coating of a stent.

Furthermore, according to the invention a coating, or combination of coatings, can be used that will

not degrade incorporated DNA.

Additionally, the second principal embodiment of the invention discussed above wherein the DNA or transformed cells are suspended in a collagen gel matrix overcomes the deficiencies of a stent having the active agents coated only on the struts. As pointed out above, the struts contact only about 10-15% of the arterial wall. Consequently, the stent of the invention wherein the interstices between the struts are filed with a collagen gel bearing DNA or transformed cells provides a much more complete treatment of the entire arterial wall.

The invention having now been fully described, it should be understood that it may be embodied in other specific forms or variations without departing from its spirit or essential characteristics. Accordingly, the embodiments described above are to be considered in all respects as illustrative and not restrictive, the scope of the invention being indicated by the appended claims rather than the foregoing description, and all changes which come within the meaning and range of equivalency of the claims are intended to be embraced therein.

We claim:

1. A method for inhibiting restenosis of blood vessels after angioplasty comprising
providing a stent comprising a lattice of interconnected struts with openings between said struts,
adhering DNA to at least a portion of said struts, said DNA coding for at least one substance capable of inhibiting restenosis of a blood vessel, and
positioning said stent having DNA adhering thereto adjacent to a wall of a lumen of a blood vessel in conjunction with an angioplasty procedure.

2. The method of claim 1, wherein said DNA is incorporated into a viral vector.

3. The method of claim 1, wherein said DNA is incorporated into a plasmid.

4. The method of claim 1, wherein said DNA is capable of transfecting cells within said blood vessel wall.

5. The method of claim 1, wherein said DNA codes for endostatin.

6. The method of claim 1, wherein said DNA codes for angiostatin.

7. The method of claim 1, wherein said DNA codes for an inhibitor of vascular endothelial growth factor (VEGF).

8. The method of claim 1, wherein said DNA codes for an inhibitor of a signal protein in a signaling cascade of vascular endothelial growth factor (VEGF).

9. The method of claim 1, wherein said DNA codes for an inhibitor of bFGF.

10. The method of claim 1, wherein said DNA codes for an inhibitor of a signal protein in a signaling cascade of bFGF.

11. The method of claim 1 wherein said stent is provided with a layer of collagen covering said struts and said openings and said DNA is adhered to said stent by embedding within said collagen layer.

11. A method for inhibiting restenosis of blood vessels after angioplasty comprising
providing a stent comprising a lattice of interconnected struts with openings between said struts,
adhering to at least a portion of said struts transfected endothelial cells capable of secreting at least one substance capable of inhibiting restenosis of a blood vessel, and
positioning said stent having transfected cells adhering thereto adjacent to a wall of a lumen of a blood vessel in conjunction with an angioplasty procedure.

12. The method of claim 11, wherein said transfected cells are capable of secreting endostatin.

13. The method of claim 11, wherein said transfected cells are capable of secreting angiostatin.

14. The method of claim 11, wherein said transfected cells are capable of secreting an inhibitor of vascular endothelial growth factor (VEGF).

15. The method of claim 11, wherein said transfected cells are capable of secreting an inhibitor of a signal protein in a signaling cascade of vascular endothelial growth factor (VEGF).

16. The method of claim 11, wherein said transfected cells are capable of secreting an inhibitor of bFGF.

17. The method of claim 11, wherein said transfected cells are capable of secreting an inhibitor of a signal protein in a signaling cascade of bFGF.

18. An intravascular stent comprising a lattice of interconnected struts with openings between said struts said stent having adhered thereto DNA coding for at least one substance capable of inhibiting restenosis of a blood vessel.

19. The intravascular stent of claim 18, wherein said stent is provided with a layer of a natural or synthetic polymer covering said struts and said openings, said polymer being compatible with DNA and said DNA being adhered to said stent by embedding within said polymer layer.

20. The intravascular stent of claim 19, wherein said polymer is collagen.

21. The intravascular stent of claim 18, wherein said DNA is incorporated into a viral vector.

22. The intravascular stent of claim 18, wherein said DNA is incorporated into a plasmid.

23. The intravascular stent of claim 18, wherein said DNA is capable of transfecting cells within said blood vessel wall.

24. The intravascular stent of claim 18, wherein said DNA codes for endostatin.

25. The intravascular stent of claim 18, wherein said DNA codes for angiostatin.

26. The intravascular stent of claim 18, wherein said DNA codes for an inhibitor of vascular endothelial growth factor (VEGF).

27. The intravascular stent of claim 18, wherein said DNA codes for an inhibitor of a signal protein in a signaling cascade of vascular endothelial growth factor (VEGF).

28. The intravascular stent of claim 18, wherein said DNA codes for an inhibitor of bFGF.

29. The intravascular stent of claim 18, wherein said DNA codes for an inhibitor of a signal protein in a signaling cascade of bFGF.

30. An intravascular stent comprising a lattice of interconnected struts with openings between said struts said stent having adhered thereto transfected endothelial cells capable of secreting at least one substance capable of inhibiting restenosis of a blood vessel.

31. The intravascular stent of claim 30, wherein said stent is provided with a layer of a natural or synthetic polymer covering said struts and said openings, said polymer being compatible with said transfected cells and said transfected cells being adhered to said stent by embedding within said polymer layer.

32. The intravascular stent of claim 31, wherein said polymer is collagen.

33. The intravascular stent of claim 30, wherein said transfected cells are capable of secreting endostatin.

34. The intravascular stent of claim 30, wherein said transfected cells are capable of secreting angiostatin.

35. The intravascular stent of claim 30, wherein said transfected cells are capable of secreting an inhibitor of vascular endothelial growth factor (VEGF).

36. The intravascular stent of claim 30, wherein said transfected cells are capable of secreting an inhibitor of a signal protein in a signaling cascade of vascular endothelial growth factor (VEGF).

37. The intravascular stent of claim 30, wherein said transfected cells are capable of secreting an inhibitor of bFGF.

38. The intravascular stent of claim 30, wherein said transfected cells are capable of secreting an inhibitor of a signal protein in a signaling cascade of bFGF.
